(19) United States
(12) Patent Application Publication
(10) Pub. No.: US 2011/0245733 A1
(43) Pub. Date: Oct. 6, 2011

(54) Methods and devices for measuring a torsion of a part of the body

(76) Inventors: Dirk David Goldbeck, Munchen (DE); Tobias Happel, Berlin (DE); Benjamin L'Henoret, Toulouse (FR)

(21) Appl. No.: 12/513,029

(22) PCT Filed: Oct. 21, 2007
(86) PCT No.: PCT/EP2007/009196

§ 371 Date: Nov. 11, 2009

(30) Foreign Application Priority Data
Nov. 2, 2006 (DE) 10 2006 051 742.3

(51) Int. Cl. A61B 5/103

(52) U.S. Cl. 600/595

(57) ABSTRACT

In a method and a device for measuring a torsion of a body with the aid of flexion sensors (B1, B2), a respective flexion effected by the torsion is detected at the flexion sensors by a first flexion signal (T1) of the first flexion sensor (B1) and a second flexion signal (T2) of the second flexion sensor (B2), and a flexion signal (CT) characteristic of the torsion is obtained by subtracting the first flexion signal from the second flexion signal or by adding the first flexion signal and the second flexion signal.

The present invention relates to methods, devices and a use of a relevant device for measuring a torsion of a body with the aid of flexion sensors.

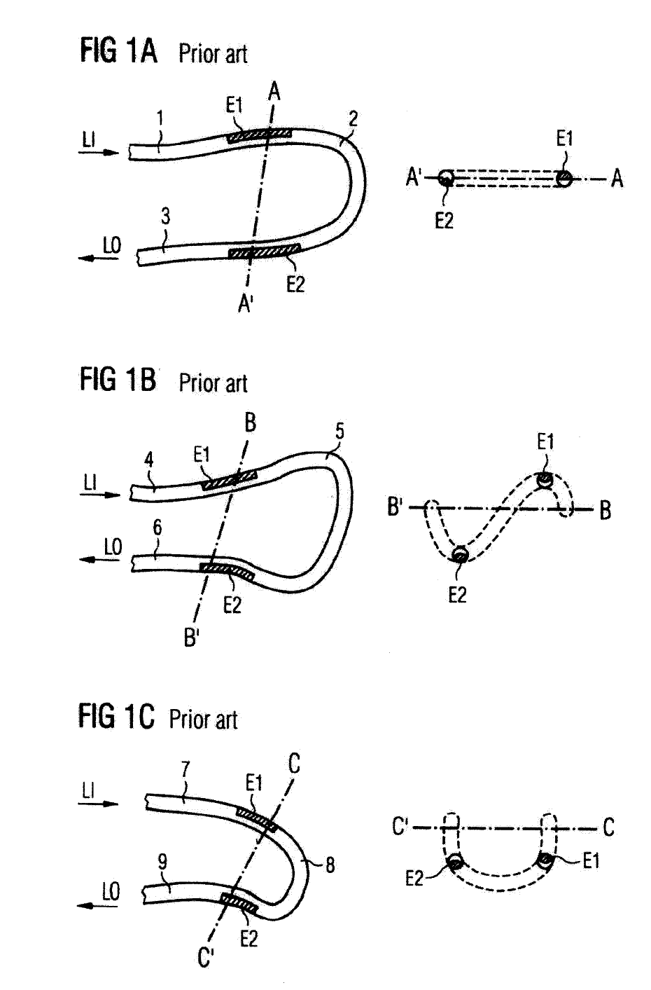

It is known from US patent [1] that a fiber-optic flexion sensor can be employed to measure flexion of a finger. In another US patent [2], as well as various fiber-optic flexion sensors, a fiber-optic sensor in accordance with FIGS. 18-22 is also specified with which a torsion is able to be measured. In this case the sensor for measurement of the torsion is embodied such that a change in transmission for a torsion is able to be measured. However the disadvantage of the sensor specified in [2] is that during a flexion, i.e. bending, a torsion is indicated. In addition, with a simultaneous flexion and torsion, the torsion is measured incorrectly. This disadvantage will be explained in greater detail with the aid of FIGS. 1A-C.

FIG. 1A shows an angled view of a loop of a fiber-optic flexion sensor, in which incoming light LI is coupled into the fiber and after passing through the loop outgoing light LO exits from the fiber. The outgoing light LO is typically processed by a photo diode or light-sensitive sensor. The ratio of incoming to outgoing light LO/LI represents an attenuation through the overall fiber loop. In the fiber are two areas E1, E2 with respective notches, with the respective transmission of the incoming light being varied depending on the radius of a flexion in the area of the respective notch. One of the areas is accommodated on the upper side and the other area on the lower side of the fiber. The position of the areas E1, E2 can be seen in the cross-section A′ - - - A. If LI=100% light is coupled in at a point (1) then for example 80% of said light is attenuated by the area E1, i.e. at the point (2) the light output is 80% of LI. Through the area E2 80% of the light output entering this area is also attenuated, so that at point (3) for the outgoing light the following then applies:

LO=80%*80%*LI=0.64*LI

FIG. 1A corresponds to the situation depicted in FIGS. 18-22 of the document [2]. In FIG. 1A the torsion sensor is in its basic position.

In the event of a torsion the areas E1, E2 twist in relation to the locations at which the light is coupled in or out. This is to be seen in the cross-section B′ - - - B. In this case for both areas E1, E2 the attenuation or the transmission of the light increases or decreases equally, depending on the direction of the torsion. If LI=100% light is coupled in at the point (4), at the point (5) there is still 90% and at point (6) 90%*90%, so that the following formula is produced for the outgoing light LO:

LO=90%*90%*LI=0.81*LI

If the torsion is in a direction opposite to this example, meaning that the attenuation increases in the area E1, E2 in relation to the basic setting, the outgoing light LO is:

LO=60%*60%*LI=0.36*LI

Since the fiber is elastic it can be assumed that the flexion radiuses produced during a torsion at the areas E1, E2 are almost identical, so that the attenuation or transmission of the light is also almost identical.

Finally in FIG. 1C the case is considered in which the loop is not subjected to a torsion but to a flexion, i.e. bending. In FIG. 1C the loop is bent downwards. This is also shown in the associated cross-section C′ - - - C. In this case the attenuation in the area E1 is greater and in the area E2 less than in the basic position in accordance with FIG. 1A. If light entering at the point (7) for LI=100% is attenuated by the area E1 by 45%, i.e. the light output at point (8) is still 55%, the light output at the point (9) can only still amount to 55% or less since the area E2 also carries out an attenuation of the light, i.e. it cannot carry out any compensation of the light attenuated by the are E1. In this example LO=0.45*LI, by which a torsion is detected, since LO/LI is smaller than the basic setting, i.e. 64%.

This thus indicates that the tension sensor proposed in [2] also measures a torsion for a flexion of the sensor even though no torsion is present. The same disadvantage is also produced if torsion and flexion, i.e. bending, occur simultaneously, with in this case the torsion being determined because of the influence of the flexion on the measurement result.

Thus the object to be achieved is to specify a method and a device which reliably determine a torsion with the aid of flexion sensors even if a flexion is also present.

This object is achieved by the features of the independent claims. Developments of the invention are to be taken from the dependent claims.

The invention relates to a method for measuring a torsion of a body with the aid of flexion sensors, with the flexion sensors being applied to a surface of the body, with the following steps:

Measuring a flexion of the flexion sensors arising from the torsion by means of a first flexion signal of the first flexion sensor and of a second flexion signal of the second flexion sensor;

Determining a characteristic flexion signal for the torsion by subtraction of the first flexion signal from the second flexion signal.

Depending on the torsion direction the flexion of for example flexion sensors accommodated on the left or right side of a torsion axis of the body is different. The two flexion sensors will therefore deliver different strongly marked flexion signals, i.e. transmission values for fiber optic flexion sensors. Linking the first flexion signal with the second flexion signal by subtraction achieves the result that only the torsion movement is measured, since a proportion of a flexion movement is eliminated by the subtraction of the two flexion signals. For example a fiber optic flexion sensor can be used as the flexion sensor, with the first flexion sensor being applied to the left and the second flexion sensor to the right of the spinal column on the back. The flexion sensors deliver different flexion signals depending on the direction of flexion. Thus for example for a flexion of the fiber optic flexion sensor in one direction a light attenuation characteristic for the flexion will increase, whereas a flexion in the other direction causes a reduction of the light attenuation.

Preferably the flexion of the first and second flexion signal occurring in a same spatial direction causes an increase or a decrease in both the first and also the second flexion signal. This enables the torsion to be measured in a reliable manner by means of the characteristic flexion signal.

In a preferable development, before the torsion is carried out, a first basic flexion signal of the first flexion sensor and a second basic flexion signal of the second flexion sensor are measured, a characteristic basic flexion signal is created by subtraction of the first and the second basic flexion signal and after the torsion is carried out the characteristic basic flexion signal is subtracted from the characteristic flexion signal determined.

The result achieved by this development is that in the rest state, i.e. before the torsion of the body, the characteristic flexion signal is zero, and thereby measurement inaccuracies of the torsion by lack of calibration of the characteristic flexion signal in the rest state are avoided.

Furthermore part of the invention is also an alternate method for measuring a torsion of a body with the aid of flexion sensor, with the flexion sensors being arranged on a surface of the body, with the following steps:

Measuring a flexion at the flexion sensors arising from the torsion in each case by means of a first flexion signal of the first flexion sensor and by means of a second flexion signal of the second flexion sensor, with the flexion of the first and second flexion sensor in a same spatial direction causing an increase of the first flexion signal and a decrease of the second flexion signal;

Determining a flexion signal characteristic for the torsion by addition of the first flexion signal and the second flexion signal.

In this case the same advantages are able to be achieved as in the explained method, with in the alternative method the generation of the flexion signal characteristic for the torsion merely being obtained by addition instead of by subtraction.

Preferably in the alternate method, before a torsion is carried out, a first basic flexion signal of the first flexion sensor and a second basic flexion signal of the second flexion sensor are measured, a characteristic basic flexion signal is created by addition of the first and of the second basic flexion signal and after the torsion is carried out the characteristic basic flexion signal is subtracted from the characteristic flexion signal determined. The benefits obtainable in this way are similar to those of the above method.

In a preferable development of the two methods flexion sensors can be used which are attached to the surface of the body almost in parallel to the axis of the torsion. This enables the flexion signals representative of the torsion and thereby the torsion itself to be measured with good accuracy. Almost parallel within the context of this invention is to be understood as allowing small deviations from the parallel arrangement to also be understood as parallel, such as ±1 cm or less for example.

If preferably the flexion sensors are used which each have a sensitive zone with a respective local spatial extension in the direction of the axis, point-type inaccuracies in the measurement of the respective flexing can be avoided, since the flexion signal represents the respective flexion via a local area of the body. The arrangement in the respective local spatial extension in the direction of the axis also means that even small torsion movements are measurable by the flexion sensors.

Preferably a torsion angle belonging to the torsion is created by means of the characteristic flexion signal based on a conversion function. This enables the torsion to be described in a simple manner by the torsion angle. The conversion function in this case specifies a relationship between the characteristic flexion signal and the associated torsion angle. This relationship is for example linear or compensates for non-linearities of the flexion sensors, e.g. shortly before the maximum flexion of the flexion sensors.

Preferably pairs of first and second flexion signals are measured over time and a time curve of the torsion is determined by generation of the respective characteristic flexion signal of respective pairs of the first and second flexion signal. This is a simple way of showing information about the torsion movement over time. This is important for example to be able to determine unfavorable movement sequences, of a patient's back for example.

In addition for both of the said methods, before determination of the characteristic flexion signal, a weighting of the respective flexion signal can be undertaken in order for example to compensate for the unequal measurement results of the flexion sensors with identical flexion.

In a preferred development of the invention the body is embodied as the back of a patient and the axis as the spinal column of the patient. Even for the calibration of the torsion of the spinal column one or more of the previous method steps can be employed to good effect.

Preferably flexion sensors are used which are embodied as fiber-optic flexion sensors. These have advantage of being neither susceptible to electromagnetic radiation nor of emitting this radiation themselves. In addition fiber-optic flexion sensors have a low weight and cheap to manufacture so that these are good to use for measuring the spinal column in a mass market.

The invention also relates to a device for measuring a torsion of a body, with the following means:

First flexion sensor and second flexion sensor which are arranged on a surface of the body;

Measurement means for measuring the respective flexion arising through the torsion at the flexion sensors by means of a first flexion signal of the first flexion sensor and by means of a second flexion signal of the second flexion sensor;

Evaluation means for determining a characteristic flexion signal for the torsion by subtraction of the first flexion signal from the second flexion signal.

Preferably the first and the second flexion sensors are arranged such that the flexion of the first and second flexion sensor occurring in a same spatial direction causes an increase or decrease of the first and of the second flexion signal.

In a development of the device the measurement means can measure a first basic flexion signal of the first flexion sensor and a second basic flexion signal of the second flexion sensor before carrying out the torsion and the evaluation means can create a characteristic basic flexion signal by subtraction of the first and the second basic flexion signal and subtract the characteristic basic flexion signal from the characteristic flexion signal determined.

The benefits obtainable from the above-mentioned device features are the same as those obtainable by the corresponding method features.

The device can also be characterized by the first and second flexion sensor respectively being embodied as a fiber optic flexion sensor with a respective sensitive zone, with the sensitive zones being embodied in a core-jacket transition of the respective flexion sensor so that sensitive zones exhibit a same spatial direction, especially perpendicular into the body or perpendicular out of the body. This specific arrangement of the sensitive zones of the flexion sensors achieves a high sensitivity in a measurement of the flexions of the flexion sensors produced by the torsion.

In addition a part of the invention is an alternative device for measuring a torsion of a body, with following means:

A first flexion sensor and a second flexion sensor which are arranged on a surface of the body, with the flexion of the first and second flexion sensor in a same spatial direction causing an increase of a first flexion signal and a decrease of a second flexion signal;

A measurement means for measuring the respective flexion arising at the flexion sensors through the torsion by means of a first flexion signal of the first flexion sensor and by means of a second flexion signal of the second flexion sensor;

Evaluation means for determining a flexion signal characteristic for the torsion by addition of the first flexion signal and the second flexion signal.

This alternate device is preferably characterized in that the measurement means, before the torsion is carried out, measures a first basic flexion signal of the first flexion sensor and a second basic flexion signal of the second flexion sensor and the evaluation means is embodied for creating a characteristic basic flexion signal by adding the first and the second basic flexion signal and for subtracting the characteristic basic flexion signal from the characteristic flexion signal determined.

Preferably the flexion sensors are accommodated on the surface of the body almost parallel to the axis of the torsion.

In a development the flexion sensors can each have a sensitive zone, with the sensitive zones being arranged with a respective local spatial extension in the direction of the axis.

In a preferred development the evaluation means create a torsion angle belonging to the torsion by means of the characteristic flexion signal based on a conversion function.

Preferably the measurement means can measure a respective pair of first and second flexion signals over time and the evaluation means can determine a time curve of the torsion by generating the respective characteristic flexion signal of respective pairs of the first and second flexion signal.

In an optional development of the respective device the body is embodied as the back of a patient and the axis as the spinal column of the patient.

Preferably flexion sensors that are embodied as fiber optic flexion sensors are used in the respective device.

The same benefits can be obtained from the above-mentioned device features of the device or alternate device as from the method features corresponding thereto.

Finally the invention comprises a use of the device or of the alternate device with at least one of the above-mentioned device features, with the device or alternate device being used for measurement of the torsion of a part of a human body, especially of the back with the spinal column as axis of the torsion.

The method or the devices can be employed in an especially efficient way for measuring the torsion of the spinal column, since the respective devices can be manufactured cost-effectively for the mass market, are well able to be worn on the patient's back and do not emit any radiation dangerous to the patient.

The invention and its developments will be explained in greater detail with reference to figures. The individual figures show:

FIG. 1 a torsion sensor known from the prior art

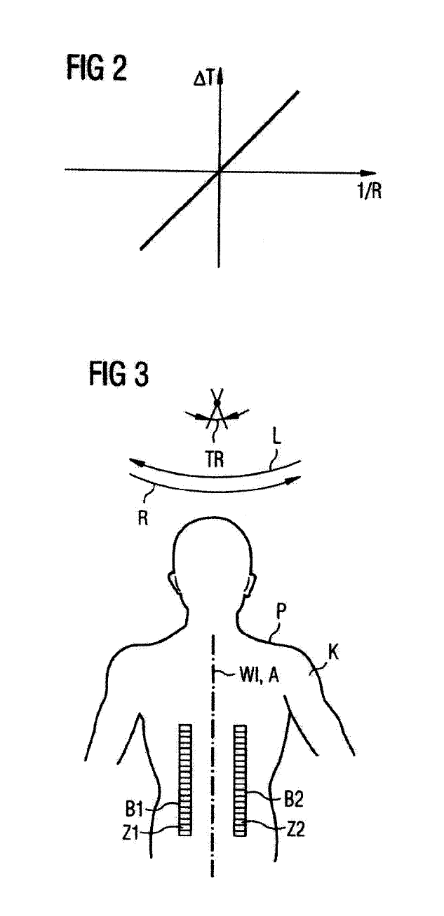

FIG. 2 a curve of a fiber optic flexion sensor with a transmission change over a flexion radius

FIG. 3 a patient, for whom a torsion of the spinal column is to be determined with the aid of two flexion sensors

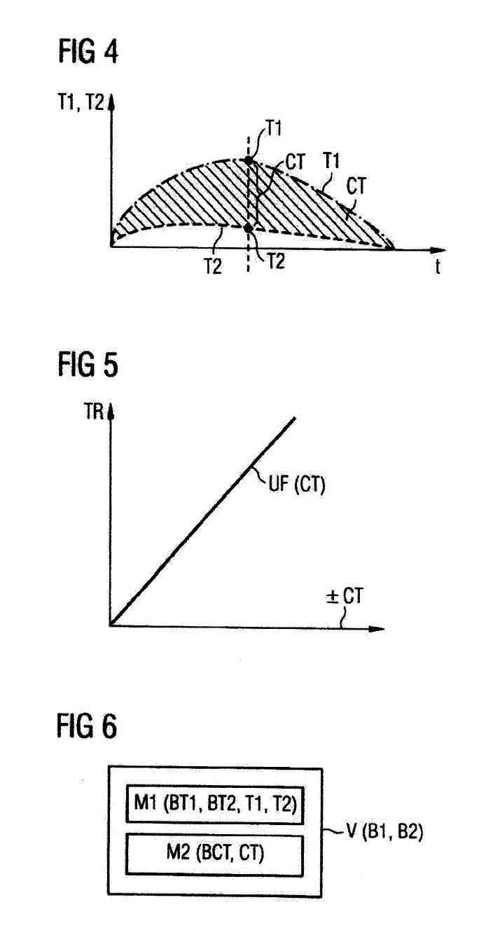

FIG. 4 a time curve of respective transmission value of the two flexion sensors

FIG. 5 a typical assignment of a torsion angle to a transmission value characteristic for the torsion

FIG. 6 a device to execute the method

Elements with same function and method of operation are provided in the figures with the same reference symbols.

The exemplary embodiments below will be explained in greater detail with reference to fiber optic flexion sensors for measurement of a torsion of a spinal column. The invention is however not restricted to this. Instead any type of flexion sensor, e.g. piezoelectric flexion sensors, can be used for measurement of any given body, e.g. a glass plate or a wooden cube. In the exemplary embodiments below for measurement of the torsion with fiber optic flexion sensors a term first or second transmission value will therefore be used as first or second flexion signal and the term characteristic transmission value as characteristic flexion signal, without the invention being restricted to this concrete embodiment.

Fiber optic flexion sensors are known from [1] for example. Here notches in the fiber are made over an area of the fiber, with a transmission of the light passing through the fiber being modified by a change in the flexion radius of the fiber in this area. If the flexion radius R is infinite, i.e. the fiber has no flexion in the area and thus 1/R=0, a basic transmission starts, see FIG. 2 in the source. If the fiber is bent in one direction, more light is coupled out than in the unbent state of the fiber so that the transmission reduces in relation to the basic transmission, see in FIG. 2 the left-hand section of the abscissa 1/R from the zero point. This means that a difference transmission AT is less than zero, ΔT<0. If the fiber is bent in the other direction the notches will be brought closer together, so that less light is coupled out in the area and thus the transmission is increased in relation to the basic transmission, see in FIG. 2 on the right-hand side of the abscissa 1/R from the zero point. This means that a difference transmission AT is greater than zero, ΔT>0. A fiber optic flexion sensor in accordance with the above explanation is understood below as the flexion sensor. The transmission is thus able to be presented as a function of the radius R if the bending of the area.

To measure the torsion of a back, i.e. of a body K, of a patient P, a first flexion sensor B1 and a second flexion sensor B2 are attached to the left and right of the spinal column WI, which represents an axis A of the torsion. In the exemplary embodiment in accordance with FIG. 3 the flexion sensors are attached respectively almost in parallel to the spinal column. Almost parallel within the context of this invention is to be understood as allowing small deviations from the parallel arrangement to also be understood as parallel, such as ±1 cm or less for example. In accordance with this embodiment the notches, i.e. sensitive zones Z1, Z2, of the two flexion sensors related to the plane of the back are either pointing in the direction of the back or in the direction away from the back, i.e. in the same spatial direction. In FIG. 3 the notches are arranged pointing away from the back.

After the patient has undertaken a torsion movement of a back and thereby of their spinal column, a respective transmission in the form of a first and second transmission value T1, T2 of the first and of the second flexion sensor B1, B2 is determined. if this determination is undertaken in an ongoing fashion over the time t, a transmission profile of the first and of the second transmission value T1, T2 is undertaken for example in accordance with FIG. 4.

To determine the torsion a transmission value CT characteristic for the torsion is determined from the first and the second transmission value T1, T2 by the following equation (1):

CT=T1(B1)−T2(B2) (1)

The amount of the characteristic transmission value CT is a measure for the strength of the torsion and a leading sign of the characteristic transmission value CT specifies the direction of the torsion. If the first flexion sensor B1 is accommodated to the left and the second flexion sensor B2 to the right of the spinal column, i.e. of the axis A of the torsion, the following table shows the direction of the torsion, with the direction being entered in FIG. 2 with corresponding arrows L or R:
Leading sign of the characteristic Direction of the transmission value CT torsion
CT>0 Left L
CT 21 0 Right R

In an optional expansion before the measurement of the torsion is undertaken a calibration of the characteristic transmission value CT is executed, so that for a position of the part of the body without torsion this indicates a value of 0. To this end a measurement of a first and second basic flexion signal or transmission value BT1, BT2 can be undertaken before the torsion, from which initially in accordance with equation (1) a calibration value i.e. a characteristic basic flexion signal is determined, i.e.

BCT=BT1(B1)−BT2(B2) (2).

For the measurement of the torsion after a torsion movement, the characteristic basic flexion signal BCT is then derived from the difference between the second and the first transmission value T2, T1, in order to obtain a value zero from a torsion in the rest state of the back. This can be described by the following equation (3):

CT=T1(B1)−T2) (B2)−BCT (3)

Furthermore the characteristic transmission value CT can be assigned a torsion angle TR, with the amount of the torsion angle TR able to be determined by the amount of the characteristic transmission value CT and the leading sign or a direction of the torsion angle TR able to be determined by the leading sign of the characteristic transmission value CT. FIG. 5 shows a typical conversion function UF, with which the amount of the characteristic transmission value CT can be converted into the amount of the torsion angle TR. This conversion function must be adapted to the specific properties of the flexion sensors used. In the example depicted in FIG. 5 the amount of the characteristic transmission value CT and the amount of the torsion angle TR are linearly dependent on one another. In general a linearization of the characteristic transmission value CT into the torsion angle can occur with the aid of the conversion function. In this example the leading sign of the torsion angle is identical to the leading sign of the characteristic transmission value CT.

As well as the computation of the torsion or of the torsion angle for a pair of a first and second transmission values the torsion and the torsion angle can also be determined over time and output on a display for example. In this case, for each pair of first and second transmission values measured over time, the torsion or the torsion angle is determined and is shown over the time in the form of a graphic or a table.

FIG. 6 shows a device for measuring a torsion of a part of the body with the aid of flexion sensors, with the flexion sensors being embodied as fiber optic flexion sensors, with a measurement means for measuring a first transmission value of the first flexion sensor and a second transmission value of the second flexion sensor after a torsion movement and with an evaluation means for determining a characteristic transmission value for the torsion by subtraction of the first transmission value from the second transmission value. Furthermore the measurement means and/or the evaluation can also be embodied to execute the method in accordance with at least one expansion. The measurement means and/or the evaluation means can be embodied in software, hardware and/or in a combination of software and hardware.

The invention has been explained in greater detail on the basis of exemplary embodiments. As well as the embodiments shown, variations are also to be understood within the framework of the invention. For example the characteristic flexion signal can be determined by subtraction of the first flexion signal from the second flexion signal. Furthermore the respective sensitive zone of the flexion sensors, i.e. that spatial direction in which a flexion is able to be measured by the flexion sensor can be oriented in the opposite spatial direction so that the characteristic flexion signal can be determined by addition of the first and second flexion signal. The variants demonstrated in the examples can also be used in combination.

### LITERATURE REFERENCES

[1] U.S. Pat. No. 5,097,252,

[2] U.S. Pat. No. 6,127,672

1. A method for measuring a torsion of a body with the aid of flexion sensors, with the flexion sensors being arranged on a surface of the body, the method comprising the following steps:
Measuring a flexion of the flexion sensors arising through the torsion by means of a first flexion signal of the first flexion sensor and by means of a second flexion signal of the second flexion sensor;
Determining a flexion signal characteristic for the torsion by subtraction of the first flexion signal from the second flexion signal.

2. The method according to claim 1, wherein,
the flexion of the first and second flexion sensor arising in a same spatial direction causes an increase or decrease of the first and of the second flexion signal.

3. The method according to claim 1, wherein,
before the torsion is carried out, a first basic flexion signal of the first flexion sensor and a second basic flexion signal of the second flexion sensor is measured,
a characteristic basic flexion signal is created by subtraction of the first and of the second basic flexion signal,
after the torsion has been carried out, the characteristic basic flexion signal is subtracted from the characteristic flexion signal determined.

4. A method for measuring a torsion of a body with the aid of flexion sensors, with the flexion sensors being arranged on a surface of the body,
the method comprising the following steps:
Measuring a flexion at the flexion sensors arising from the torsion in each case by means of a first flexion signal of the first flexion sensor and by means of a second flexion signal of the second flexion sensor, with the flexion of the first and second flexion sensor in a same spatial direction causing an increase of the first flexion signal and a decrease of the second flexion signal;
Determining a flexion signal characteristic for the torsion by addition of the first flexion signal and the second flexion signal.

5. The method according to claim 4, wherein,
before the torsion is carried out a first basic flexion signal of the first flexion sensor and a second basic flexion signal of the second flexion sensor is measured,
a characteristic basic flexion signal is created by addition of the first and of the second basic flexion signal,
after the torsion has been carried out the characteristic basic flexion signal is subtracted from the characteristic flexion signal determined.

6. The method according to claim 4, wherein
flexion sensors are used which are attached to the surface of the body almost in parallel for axis of the torsion.

7. The method according to claim 4, wherein
flexion sensors are used which have a respective sensitive zone with a respective local spatial extension in the direction of the axis.

8. The method according to claim 4, wherein
a torsion angle belonging to the torsion is created by means of the characteristic flexion signal on the basis of a conversion function.

9. The method according to claim 4, wherein
a respective pair of first and second flexion signals are measured over time, a time curve of the torsion is determined by generation of the respective characteristic flexion signal of respective pairs of the first and second flexion signal.

10. The method according to claim 4, wherein
the body is embodied as a patient's back and the axis as the patient's spinal column.

11. The method according to claim 4, wherein
flexion sensors are used that are embodied as fiber optic flexion sensors.

12. The method according to claim 4, wherein
before the determination of the characteristic flexion signal a weighting of the respective flexion signals is undertaken.

13. A device for measuring a torsion of a body, comprising:
First flexion sensor and second flexion sensor, which are arranged on a surface of the body;
A measurement unit for measuring the respective flexion at the flexion sensor arising from the torsion by means of a first flexion signal of the first flexion sensor and by means of a second flexion signal of the second flexion sensor;
An evaluation unit for determining a characteristic flexion signal for the torsion by subtraction of the first flexion signal from the second flexion signal.

14. The device according to claim 13, wherein,
the first and the second flexion sensors are arranged such that the flexion of the first and second flexion sensor arising in a same spatial direction causes an increase or decrease of the first and of the second flexion signal.

15. The device according to claim 13, wherein
before the torsion is carried out, the measuring unit is operable to measure a first basic flexion signal of the first flexion sensor and a second basic flexion signal of the second flexion sensor; and wherein
the evaluation unit is operable to create a characteristic basic flexion signal by subtraction of the first and the second basic flexion signal and further operable to subtract the characteristic basic flexion signal from the determined characteristic flexion signal.

16. The device according to claim 13, wherein
the first and second flexion sensor are embodied respectively as fiber optic flexion sensors with a respective sensitive zone, with the sensitive zones being embodied at the core jacket transition of the respective flexion sensor such that the sensitive zones point in a same spatial direction, or perpendicular into the body or perpendicular out of the body.

17. A device for measuring a torsion of a body, comprising:
a first flexion sensor and second flexion sensor, that are arranged on a surface of the body, with the flexion of the first and second flexion sensor in a same spatial direction causing an increase of a first flexion signal and a decrease of a second flexion signal;
A measurement unit for measuring the respective flexion at the flexion sensor occurring through the torsion by means of a first flexion signal of the first flexion sensor and by means of a second flexion signal of the second flexion sensor;
An evaluation unit for determining a flexion signal characteristic for the torsion by addition of the first flexion signal and the second flexion signal.

18. The device according to claim 17, wherein
before the torsion is carried out, the measuring unit is operable to measure a first basic flexion signal of the first flexion sensor and a second basic flexion signal of the second flexion sensor; and wherein
the evaluation unit is operable to create a characteristic basic flexion signal by adding the first and the second basic flexion signal and further operable to subtract the characteristic basic flexion signal from the determined characteristic flexion signal.

19. The device according to claim 17, wherein
the flexion sensors are arranged on the surface of the body almost in parallel to the axis of the torsion.

20. The device according to claim 17, wherein
the flexion sensors each have a sensitive zone, with the sensitive zone with a respective local spatial extension being arranged in the direction of the axis.

21. The device according to claim 17, wherein
The evaluation unit is operable to create a torsion angle belonging to the torsion by means of the characteristic flexion signal based on a conversion function.

22. The device according to claim 17, wherein
the measurement unit is operable to measure a respective pair of first and second flexion signals over time,
the evaluation unit is operable to determines a time curve of the torsion by generating the respective characteristic flexion signal of respective pairs of the first and second flexion signal.

23. The device according to claim 17, wherein
the body is embodied as a patient's back and the axis as the patient's spinal column.

24. The device according to claim 17, wherein
flexion sensors are used that are embodied as fiber optic flexion sensors.

25. A method comprising the step of using of a device according to claim 17, comprising the step of
using the device for measurement of the torsion of a back of a human body with a spinal column as the axis of the torsion or of another part of the human body.

26. The method according to claim 1, wherein
flexion sensors are used which are attached to the surface of the body almost in parallel for axis of the torsion.

27. The method according to claim 1, wherein
flexion sensors are used which have a respective sensitive zone with a respective local spatial extension in the direction of the axis.

28. The method according to claim 4, wherein
a torsion angle belonging to the torsion is created by means of the characteristic flexion signal on the basis of a conversion function.

29. The method according to claim 1, wherein
a respective pair of first and second flexion signals are measured over time,
a time curve of the torsion is determined by generation of the respective characteristic flexion signal of respective pairs of the first and second flexion signal.

30. The method according to claim 1, wherein
the body is embodied as a patient's back and the axis as the patient's spinal column.

31. The method according to claim 1, wherein
flexion sensors are used that are embodied as fiber optic flexion sensors.

32. The method according to claim 1, wherein
before the determination of the characteristic flexion signal a weighting of the respective flexion signals is undertaken.

33. The device according to claim 13, wherein
the flexion sensors are arranged on the surface of the body almost in parallel to the axis of the torsion.

34. The device according to claim 13, wherein
the flexion sensors each have a sensitive zone, with the sensitive zone with a respective local spatial extension being arranged in the direction of the axis.

35. The device according to claim 13, wherein
The evaluation unit is operable to create a torsion angle belonging to the torsion by means of the characteristic flexion signal based on a conversion function.

36. The device according to claim 13, wherein
the measurement unit is operable to measure a respective pair of first and second flexion signals over time,
the evaluation unit is operable to determine a time curve of the torsion by generating the respective characteristic flexion signal of respective pairs of the first and second flexion signal.

37. The device according to claim 13, wherein
the body is embodied as a patient's back and the axis as the patient's spinal column.

38. The device according to claim 13, wherein
flexion sensors are used that are embodied as fiber optic flexion sensors.

39. A method comprising the step of using of a device according to claim 13, comprising the step of
using the device for measurement of the torsion of a back of a human body with a spinal column as the axis of the torsion or of another part of the human body.
